(19) United States
(12) Patent Application Publication
(10) Pub. No.: US 2001/0005722 A1
(43) Pub. Date: Jun. 28, 2001

(54) endo-N-(9-methyl-9-azabicyclo[3.3.1]non-3-yl)-1-methyl-1h-indazole-3-carboxamide hydrochloride

(76) Inventor: Susan James, Haverhill (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 09/548,959

(22) Filed: Apr. 13, 2000

Related U.S. Application Data
(63) Continuation of application No. 09/103,432, filed on Jun. 24, 1998, now abandoned.
(60) Provisional application No. 60/050,664, filed on Jun. 24, 1997.
(60) Provisional application No. 60/050,738, filed on Jun. 25, 1997.

(51) Int. Cl. C07D 401/00; C07D 405/00; C07D 409/00; C07D 411/00; C07D 413/00; A61K 31/44

(52) U.S. Cl. 514/299; 514/304; 546/112; 546/126

(57) ABSTRACT

Invented are improved solid dosage formulations of endo-N-(9-methyl-9-azabicyclo[3.3.1.]non-3-yl)-1-methyl-1H-indazole-3-carboxamide hydrochloride. Also invented is an imporved method of administering an anti-emeticly effective amount of the compound endo-N-(9-methyl-9-azabicyclo[3.3.1.]non-3-yl)-1-methyl-1H-indazole-3-carboxamide hydrochloride.

This invention relates to improved formulations of endo-N-(9-methyl-9-azabicyclo[3.3.1.]non-3-yl)-1-methyl-1H-indazole-3-carboxamide hydrochloride. The compound is represented by Structure I:

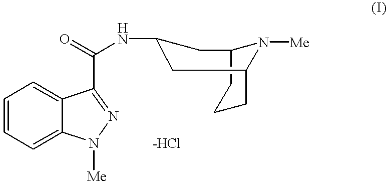

The formulations of this invention are useful as anti-emetics, particularly in the treatment of cytotoxic agent induced emesis.

### DETAILED DESCRIPTION OF THE INVENTION

Endo-N-(9-methyl-9-azabicyclo[3.3.1.]non-3-yl)-1-methyl-1H-indazole-3-carboxamide hydrochloride is a compound which is disclosed and claimed, along with pharmaceutically acceptable salts, hydrates and solvates thereof, as being useful as an anti-emetic, particularly in the treatment of cytotoxic agent induced emesis, in U.S. Pat. No. 4,886,808, the entire disclosure of which is hereby incorporated by reference. Endo-N-(9-methyl-9-azabicyclo[3.3.1.]non-3-yl)-1-methyl-1H-indazole-3-carboxamide hydrochloride can be prepared by methods such as described in U.S. Pat. No. 4,886,808. Endo-N-(9-methyl-9-azabicyclo[3.3.1.]non-3-yl)-1-methyl-1H-indazole-3-carboxamide hydrochloride is commercially available under the trade name Kytril and is also known by the generically as granisetron hydrochloride.

As indicated in the Physicians' Desk Reference®, 1997 edition, published by Medical Economics Company, Inc. at Montvale, N.J., a solid dosage form of endo-N-(9-methyl-9-azabicyclo[3.3.1.]non-3-yl)-1-methyl-1H-indazole-3-carboxamide hydrochloride is commercially available as a film-coated tablet containing 1.12 mg of granisetron hydrochloride equivalent to granisetron 1 mg and is administered twice a day. Alternative dose regimens for the oral administration of granisetron hydrochloride are described in Ettinger, D. et al., Cancer: 78; 1996: 144-151.

All publications, including but not limited to patents and patent applications, cited in this specification are herein incorporated by reference as though fully set forth.

Numerous advantages would be realized if a suitable once a day dosage form of endo-N-(9-methyl-9-azabicyclo[3.3.1.]non-3-yl)-1-methyl-1H-indazole-3-carboxamide hydrochloride could be prepared. The advantages of a once a day dosage form of endo-N-(9-methyl-9-azabicyclo[3.3.1.]non-3-yl)-1-methyl-1H-indazole-3-carboxamide hydrochloride include: ease of administration for the patient, the patient will have to remember to take the medication only once a day instead of two times a day, fewer pills to count thereby decreasing the chances of over or under dosing and more cost effective to produce and transport.

It has now surprisingly been found that suitable once a day dosage forms of endo-N-(9-methyl-9-azabicyclo[3.3.1.]non-3-yl)-1-methyl-1H-indazole-3-carboxamide hydrochloride can readily be prepared. Suitably, the once a day tablet formulations of endo-N-(9-methyl-9-azabicyclo[3.3.1.]non-3-yl)-1-methyl-1H-indazole-3-carboxamide hydrochloride will exhibit a desirable dissolution profile, specified hardness range and compression weight.

While endo-N-(9-methyl-9-azabicyclo[3.3.1.]non-3-yl)-1-methyl-1H-indazole-3-carboxamide hydrochloride may be effectively administered by any one of a number of ways, administering this compound once a day in a solid dosage form containing about 2 mg of the active substance, endo-N-(9-methyl-9-azabicyclo[3.3.1.]non-3-yl)-1-methyl-1H-indazole-3-carboxamide, has the added advantages of; ease of administration for the patient, the patient will have to remember to take the medication only once a day instead of two times a day, may lead to fewer pills to count thereby decreasing the chances of over or under dosing and more cost effective to produce and transport.

### Dissolution Testing

The tablets containing about 2.24 mg of endo-N-(9-methyl-9-azabicyclo[3.3.1.]non-3-yl)-1-methyl-1H-indazole-3-carboxamide hydrochloride (equal to about 2 mg of endo-N-(9-methyl-9-azabicyclo[3.3.1.]non-3-yl)-1-methyl-1H-indazole-3-carboxamide) and prepared as described herein were tested in deionized water following the USP paddle method (Apparatus 2, 50 rpm, at 37° C.). The percentage of endo-N-(9-methyl-9-azabicyclo [3.3.1.]non-3-yl)-1-methyl-1H-indazole-3-carboxamide hydrochloride released was determined by HPLC with UV detection. Tablets prepared according to this invention demonstrated greater than 75% of the nominal drug content being dissolved in 45 minutes.

### Hardness

Tablets prepared according to this invention were Schleuniger hardness tested. Preferred tablets were within a hardness range of 4.5 to 7.5 kP, advantageously 5 to 7 kP, most preferred 6 kP.

The present invention includes within its scope pharmaceutical compositions comprising about 2.24 mg of endo-N-(9-methyl-9-azabicyclo[3.3.1.]non-3-yl)-1-methyl-1H-indazole-3-carboxamide hydrochloride, in association with a pharmaceutically acceptable carrier. The compound of this invention is administered orally and can be formulated in dosage forms appropriate for such administration including capsules, tablets, pills, powders and granules, preferably tablets. In these solid dosage forms, the active compound is admixed with at least one inert diluent such as sucrose, lactose or starch. The oral dosage forms can also comprise, as is normal practice, additional substances other than inert diluents, e.g., lubricating agents such as magnesium stearate, glidants such as colloidal silicone dioxide, antioxidants such as butylated hydizoxy toluene. In the case of capsules, tablets and pills, the dosage forms may also comprise buffering agents. Tablets and pills can additionally be prepared for a sustained release or may be prepared with enteric coatings. Additionally, multiple tablets can be given once a day so long as the total amount of endo-N-(9-methyl-9-azabicyclo[3.3.1.]non-3-yl)-1-methyl-1H-indazole-3-carboxamide being administered is about 2 mg.

All of the pharmaceutical excipients utlizied herein are known and commercially available.

The following examples further illustrate the present invention. The examples are not intended to limit the scope of the invention as defined hereinabove and as claimed below.

### EXAMPLE 1

### Preparation of endo-N-(9-methyl-9-azabicyclo[3.3.1.]non-3-yl)-1-methyl-1H-indazole-3-carboxamide hydrochloride—2 mg tablets

Endo-N-(9-methyl-9-azabicyclo[3.3.1.]non-3-yl)-1-methyl-1H-indazole-3-carboxamide hydrochloride—2 mg tablets were prepared as indicated below using ingredients in the following ratios.

i) endo-N-(9-methyl-9-azabicyclo[3.3.1.]non-3-yl)-1-methyl-1H-indazole-3-carboxamide hydrochloride—equivalent to 2 mg endo-N-(9-methyl-9-azabicyclo[3.3.1.]non-3-yl)-1-methyl-1H-indazole-3-carboxamide per tablet;

ii) microcrystalline cellulose—40 mg per tablet;

iii) hydroxypropyl methylcellulose—8 mg per tablet;

iv) sodium starch glycollate—10 mg per tablet;

v) magnesium stearate—2 mg per tablet;

vi) lactose—to 200 mg per tablet; and

vii) film coat—6 mg per tablet.

The intragranular excipients (lactose, microcrystalline cellulose, hydroxypropyl methylcellulose, sodium starch glycollate) were uniformly blended in a high shear mixer. The uniformly blended excipients were wet granulated with a solution of endo-N-(9-methyl-9-azabicyclo[3.3.1.]non-3-yl)-1-methyl-1H-indazole-3-carboxamide hydrochloride in water using a high shear mixer. The wet granules were milled and dryed using a fluid bed dryer. The dryed granules were blended with magnesium stearate in a high shear mixer. The resulting pharmaceutical blend was compressed into tablets using a standard tablet press. The prepared tablets were aqueous film coat in a standard film coating machine.

While the preferred embodiments of the invention are illustrated by the above, it is to be understood that the invention is not limited to the precise instructions herein disclosed and that the right to all modifications coming within the scope of the following claims is reserved.

What is claimed is:

1. A method of administering an anti-emeticly effective amount of the compound endo-N-(9-methyl-9-azabicyclo[3.3.1.]non-3-yl)-1-methyl-1H-indazole-3-carboxamide hydrochloride which comprises administering about a 2 mg oral dose of endo-N-(9-methyl-9-azabicyclo[3.3.1.]non-3-yl)-1-methyl-1H-indazole-3-carboxamide once a day.

2. The compound endo-N-(9-methyl-9-azabicyclo[3.3.1.]non-3-yl)-1-methyl-1H-indazole-3-carboxamide hydrochloride in a pharmaceutical tablet formulation for oral administration comprising about a 2 mg of endo-N-(9-methyl-9-azabicyclo[3.3.1.]non-3-yl)-1-methyl-1H-indazole-3-carboxamide.

3. A pharmaceutical tablet formulation as described in claim 2 in which 75% of the active compound, endo-N-(9-methyl-9-azabicyclo[3.3.1.]non-3-yl)-1-methyl-1H-indazole-3-carboxamide hydrochloride, is dissolved in 45 minutes when subjected to dissolution testing using standard USP criteria.

4. A pharmaceutical tablet formulation as described in claim 3 in which 75% of the active compound, endo-N-(9-methyl-9-azabicyclo[3.3.1.]non-3-yl)-1-methyl-1H-indazole-3-carboxamide hydrochloride, is dissolved in 30 minutes when subjected to dissolution testing using standard USP criteria.

5. A pharmaceutical tablet formulation as described in claim 4 which comprises
i) endo-N-(9-methyl-9-azabicyclo[3.3.1.]non-3-yl)-1-methyl-1H-indazole-3-carboxamide hydrochloride—equivalent to about 2 mg endo-N-(9-methyl-9-azabicyclo[3.3.1.]non-3-yl)-1-methyl-1H-indazole-3-carboxamide per tablet;
ii) microcrystalline cellulose—about 40 mg per tablet;
iii) hydroxypropyl methylcellulose—about 8 mg per tablet;
iv) sodium starch glycollate—about 10 mg per tablet;
v) magnesium stearate—about 2 mg per tablet;
vi) lactose—to 200 mg per tablet; and
vii) film coat—about 6 mg per tablet.

6. A pharmaceutical tablet formulation as described in claim 5 which exhibits a hardness of from about 4.5 kP to 7.5 kP.
